(19) United States
(12) Patent Application Publication
(10) Pub. No.: US 2010/0136520 A1
(43) Pub. Date: Jun. 3, 2010

(54) DETECTING HEPATITIS B VIRUS

(75) Inventors: Paul F. Coleman, Antioch, IL (US); Akhtar X. Ali, Green Oaks, IL (US); Tanseem Ali, Green Oaks, IL (US)

Correspondence Address: PAUL D. YASGER;ABBOTT LABORATORIES, 100 ABBOTT PARK ROAD, DEPT. 377/AP6A, ABBOTT PARK, IL, 60064-6008, US

(73) Assignee: ABBOTT LABORATORIES, Abbott Park, IL (US)

(21) Appl. No.: 12/629,901

(22) Filed: Dec. 3, 2009

Related U.S. Application Data
(63) Continuation-in-part of application No. 12/209,054, filed on Sep. 11, 2008, now abandoned.
(60) Provisional application No. 60/972,137, filed on Sep. 13, 2007.

(51) Int. Cl. C12Q 1/70

(52) U.S. Cl. 435/5

(57) ABSTRACT

This invention is related to methods for detecting hepatitis B virus by determining the level of hepatitis B virus surface antigen protein. This invention is also related to methods for detecting mutant hepatitis B virus surface antigen protein and kits for detecting hepatitis B virus.

The subject application is a continuation-in-part of U.S. Ser. No. 12/209,054 filed on Sep. 11, 2008 which claims priority to U.S. provisional application Ser. No. 60/972,137 filed on Sep. 13, 2007, herein incorporated in its entirety by reference.

### FIELD OF THE INVENTION

This invention relates to methods for detecting hepatitis B viral antigens and kits related thereto.

### BACKGROUND OF THE INVENTION

The hepatitis B virus (HBV) is estimated to have infected over 2 billion people worldwide. HBV is known to cause a variety of disease states from mild subclinical infection to chronic active and fulminant hepatitis. Over 400 million people, especially children and the elderly, are chronically infected with HBV. The hepatitis B virus is 100 times more infectious than the AIDS virus, yet it can be prevented with vaccination. A key strategy in controlling HBV infection is universal vaccination as well as early detection and treatment of infected individuals. Accordingly, HBV diagnostic assays have focused on improved and accurate detection of HBV viral antigens.

The HBV genome is a circular, partially double stranded DNA sequence of approximately 3200 basepairs which code for at least five open reading frames (ORF) (Tiollais et al., Nature 317:489-495 (1985)). There are four genes (polymerase (P), surface (S), core (C), and (X)); the polymerase gene overlaps the surface gene and also partially overlaps the X and core genes. These four genes produce seven proteins; the product of the surface gene consists of three proteins that have different initiation sites but the same termination site. These three proteins (i.e., small (S), middle (M), and large (L) surface antigen (HBsAg) therefore all contain the S-HBsAg gene sequence of 226 amino acids (Gerlich et al. in Viral Hepatitis and Liver Disease, Hollinger et al., eds., Williams-Wilkens, Baltimore, Md., pages 121-134 (1991)). The M-HBsAg contains the 55 amino acid PreS2 sequence and the S sequence for a total length of 281 amino acids. The L-HBsAg protein contains the 108 amino acid PreS1 sequence plus the PreS2 and S sequences for a total length of 389 amino acids. In addition, each of the three envelope proteins exhibits different degrees of glycosylation. These three proteins are expressed at different ratios with S-HBsAg compromising approximately 95% of the total protein and assemble to form the outer capsid of the HBV viron. (See FIG. 1 and FIG. 2). L-HBsAg, M-HBsAg, and S-HBsAg also assemble in a similar form to produce an incomplete viral particle. HBV surface antigen assays detect both morphological forms—virons and particles.

The core gene encodes the nucleocapsid protein, hepatitis B core antigen (HBcAg). Immediately upstream of the core gene is the precore region. The first 19 amino acids of the precore region serve as a signal for membrane translocation and eventual secretion of the precore gene product, the hepatitis B e antigen (HBeAg).

Similar to the Human Immunodeficiency Virus (HIV), HBV uses reverse transcriptase (RT) as an essential step in the replication cycles. However, RT has poor proofreading ability, thereby leading to a high rate of nucleotide misincorporation. Calculations suggest that this error-prone replication leads to one point replacement, deletion or insertion per 1000 to 100,000 nucleotides copied (Carman et al., Lancet 341:349-353 (1993)). Variability in HBV surface antigen was first described using classical subtyping studies Courouce et al., Bibliotheca Haematologica 42:1 (1976)). There are eight known genotypes of HBV that are thought to have arisen from replication variation (Norder et al., Intervirology 47:289-309 (2004)). The HBV envelope regions encompassing PreS1 and PreS2 and the S “a” determinant are exposed on the surface of the viral particle and are therefore expected to be targets of immune surveillance (Gerlich et al., supra). Some surface antigen mutants previously described have significantly affected the antigenicity of the “a” determinant that contains both common and group-specific determinants (Carman et al., Gastroenterology 102:711-719 (1992)). The “a” determinant is located between amino acids 100-160 of S-HBsAg and presents a complex conformational epitope, which is stabilized by disulfide bonding between highly conserved cysteine residues. The “a” determinant immunoreactivity can be partially mimicked using cyclic synthetic peptides. Further, although the “a” determinant had been traditionally defiled by reactivity to polyclonal antisera, the use of monoclonal antibody has shown that the “a” determinant consists of at least five partially overlapping epitopes (Peterson et al., J. Immunol. 132:920-927 (1984)). The most common surface antigen mutant described in the literature is a single nucleotide substitution leading to the substitution of glycine at amino acid position 145 of S-HBsAg with arginine (G-R 145). This G-R 145 mutation destroys some, but not all, “a” determinant epitopes.

Additionally, other mutations in the “a” determinant result in loss of subtypic or type-specific determinants y/d and w/r. Also the emergence of gross deletions and point mutations in the PreS1/PreS2 region suggest that the product of the surface gene is under immune selection in chronically infected patients. Further, HBV mutants that cannot replicate because of deletions in the S, C or P genes have been noted in plasma from HBV carriers. All co-exist with HBV forms which are replication competent.

Okamoto et al. have demonstrated that mutant genomes with gross deletions in the PreS/S, C and P genes derived from plasma or asymptomatic carriers may be complemented in transient expression systems with hepatoma cells (Okamoto et al., Pediatric Research 32:264-268 (1992)). In fact, the suggestion has been made that HBV mutants acting as defective interfering particles may attenuate wildtype virus replication and thereby help maintain persistence of the infection.

Accordingly, HBV is capable of evading immune surveillance and vaccination regimens via mutations in the surface proteins, including S-HBsAg. Furthermore, because some methods of HBV detection depend on monitoring epitopes within the envelope proteins by using S-HBsAg antibodies, highly mutated HBV may also escape detection. There is hence a continued need in the art for methods and compositions for detecting HBV.

All U.S. patents and publications are incorporated in their entirety herein by reference.

### SUMMARY OF THE INVENTION

Provided herein is a method for detecting wildtype or mutant forms of hepatitis B in a sample, which may comprise providing a sample and contacting the sample with a first antibody to a middle hepatitis B surface protein (“M-HBsAg”) for a time and under conditions sufficient to a form M-HBsAg/first antibody complex. The identification of a M-HBsAg/first antibody complex may be indicative of the detection of hepatitis B in the sample. The first antibody may not cross-react with S-HBsAg. Amino acids 1-55 of M-HBsAg may comprise an epitope recognized by the first antibody. A second antibody that is capable of binding small hepatitis B surface protein (“S-HBsAg”) may be also used for a time and under conditions sufficient to form S-HBsAg/second antibody complexes to determine the presence of hepatitis B in the sample. Amino acids 100-160 of S-HBsAg comprise an epitope recognized by the second antibody. The second antibody may be used in conjunction with the first antibody.

Also provided herein is a first method for detecting a mutant S-HBsAg in a sample. Levels of M-HBsAg/first antibody complexes and S-HBsAg/second antibody complexes in a sample may be determined and compared. A first antibody that is capable of binding M-HBsAg may be used to determine the level of M-HBsAg. A second antibody that is capable of binding S-HBsAg may be used to determine the level of S-HBsAg. The identification of a reduced level of S-HBsAg/second antibody complexes, as compared to M-HBsAg/first antibody complexes, may be indicative of mutant S-HBsAg in the sample. The first antibody may not cross-react with S-HBsAg. Amino acids 1-55 of M-HBsAg may comprise an epitope recognized by the first antibody. Amino acids 100-160 of S-HBsAg comprise an epitope recognized by the second antibody.

Provided herein is a second method for detecting a mutant S-HBsAg in a sample, which may comprise providing a sample and contacting the sample with an antibody to a S-HBsAg. The identification of a reduced level of antibody/S-HBsAg complex, as compared to a control, may be indicative of a mutant S-HBsAg in the sample.

The method may further comprise an alternate method for determining the level of wildtype or mutant forms of HBsAg in the sample. The method may comprise providing a sample and contacting a first antibody that is capable of binding S-HBsAg for a time and under conditions sufficient to form a S-HBsAg/first antibody complex may be used to determine the level of HBsAg. The first antibody may not recognize mutant forms of S-HBsAg and a reduced level of S/HBs/first antibody complex in comparison to a control indicates detection of a mutant S-HBsAg in the test sample. The method may further comprising providing a test sample and contacting a second antibody that is capable of binding S-HBsAg for a time and under conditions sufficient to form a S-HBAg/second antibody complex may be used to determine the level of S-HBsAg. A difference in the ratio of the level of S-HBsAg detected by the first antibody to the level of S-HBsAg detected by the second antibody compared to a predetermined ratio level may indicate detection of a mutant S-HBsAg in the test sample.

Further provided herein is a kit for detecting hepatitis B. The kit may comprise at least one antibody that is capable of binding to M-HBsAg and at least one antibody that is capable of binding to S-HBsAg Amino acids 1-55 of M-HBsAg may comprise an epitope recognized by at least one antibody capable of binding M-HBsAg. The antibody or antibodies that cross react with M-HBsAg may not cross-react with S-HBsAg Amino acids 100-160 of S-HBsAg may comprise an epitope recognized by at least one antibody capable of binding M-HBsAg. The antibody or antibodies that cross react with S-HBsAg may not cross react with M-HBsAg.

### BRIEF DESCRIPTION OF THE DRAWINGS

FIG. 1 is a schematic depiction of a HBV virion, including the HBV envelope protein constituents L-HBsAg, M-HBsAg, and S-HBsAg.

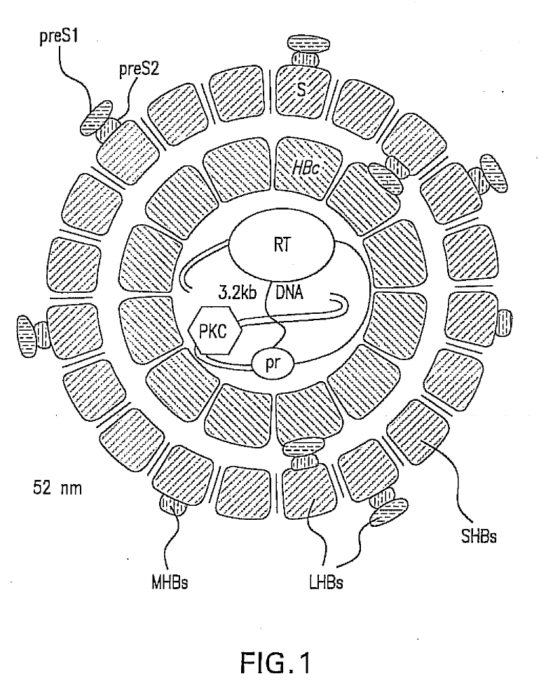

FIG. 2 shows a schematic depiction of the HBV surface gene open reading frame and the initiation sites for the preS1, preS2 and S regions. FIG. 2 also shows a schematic of the HBV surface proteins L-HBsAg, M-HBsAg, and S-HBsAg transcribed from the preS1, preS2 and S initiation sites, respectively.

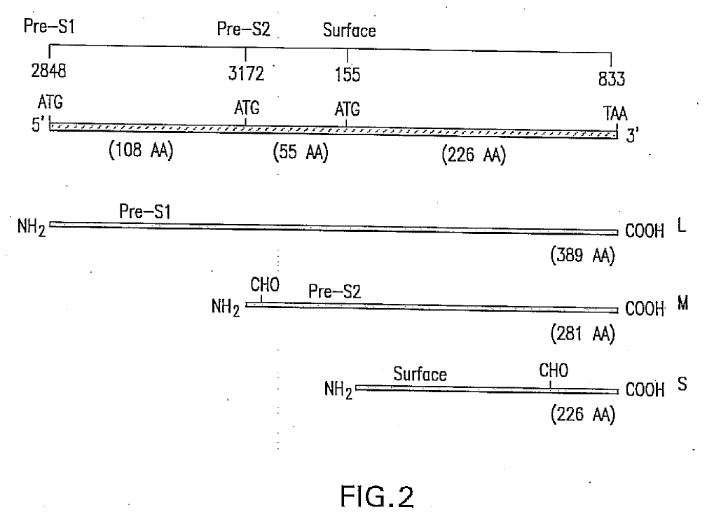

FIG. 3 shows a schematic representation of known naturally-occurring mutations between amino acids 121-124 in S-HBsAg. The mutations affect an epitope capable of being bound by Abbott anti-HBs monoclonal antibody H166.

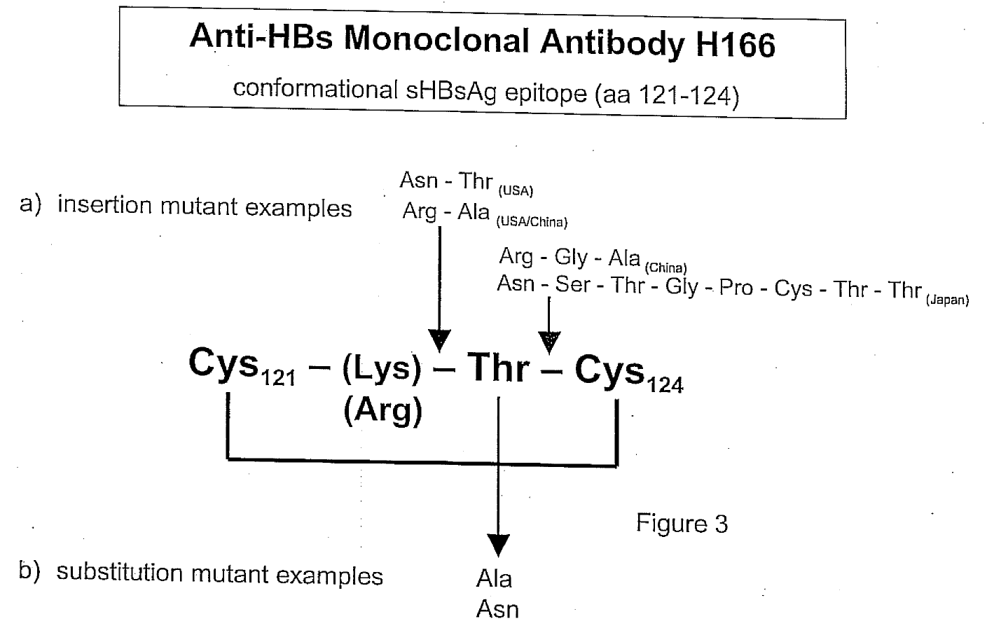

FIG. 4 shows the relative capture avidity of anti-HBs monoclonal antibodies 116-34, H166, H57, H53, H40, and H35.

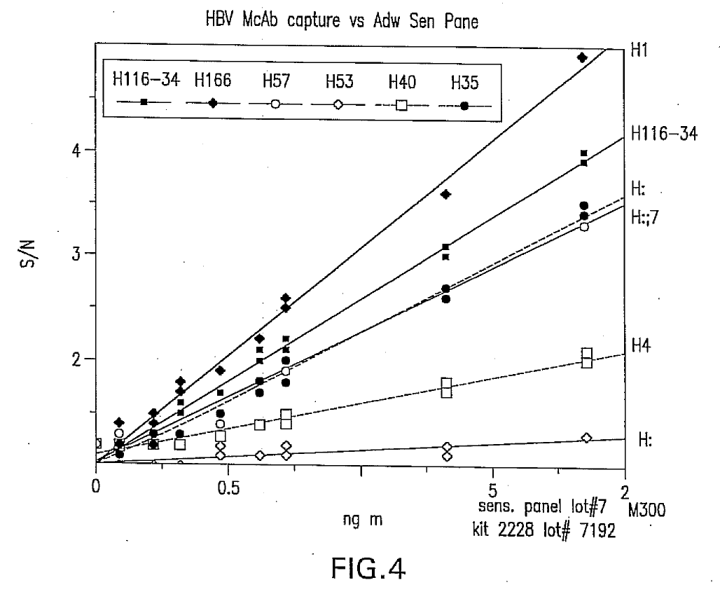

FIG. 5 shows a comparison between H166, 116-34, and H53 in detecting HBV in clinical specimens collected from a Canadian population.

FIG. 6 shows a comparison the ability of 116-34, H166, H57, H35, and H53 to detect HBV in serum specimens containing known HBV mutants.

FIG. 7 shows a comparison of the ability of H166, H40, H57, H35, H53, 116-34, and a combination of H40, H57, and H116 to detect wildtype and mutant HBV samples.

### DETAILED DESCRIPTION

The inventors have made the surprising discovery that anti-HBs detecting M-HBsAg alone, or in combination with anti-HBs detecting S-HBsAg, provides a more sensitive and accurate means of diagnosing HBV infection. This is especially significant since M-HBsAg comprises only a small portion of the capsid total protein. Currently available methods for detecting HBV are accomplished by utilizing anti-HBs directed against the more plentiful S-HBsAg alone. However, some commercially available assays utilizing these anti-HBs antibody reagents have been found to be unable to detect S-HBsAg in some patients, despite a known HBV infection that can be detected by other means. This false negative result may be due to inability of the kit reagents to detect S-HBsAg because the HBV strain harbors a mutation or series of mutations that alter the S-HBsAg sequence. These mutations can affect S-HBsAg epitopes so that the anti-HBs monoclonal antibody reagents can no longer detect HBV infections (Coleman et al., J Med. Virol. 59:19-24 (1999)). Epitope alterations are localized to S-HBsAg and do not extend significantly to the M-HBsAg domain.

### 1. DEFINITIONS

The terminology used herein is for the purpose of describing particular embodiments only and is not intended to be limiting. As used in the specification and the appended claims, the singular forms “a,” “an” and “the” include plural referents unless the context clearly dictates otherwise.

For recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are explicitly contemplated.

a. Antibody

“Antibody” as used herein may mean an antibody of classes IgG, IgM, IgA, IgD or IgE, or fragments or derivatives thereof, including Fab, F(ab′)2, Fd, and single chain antibodies, diabodies, bispecific antibodies, bifunctional antibodies and derivatives thereof. The antibody may be a monoclonal antibody, polyclonal antibody, affinity purified antibody, or mixtures thereof which exhibits sufficient binding specificity to a desired epitope or a sequence derived therefrom. The polyclonal antibody may be of mammalian origin, such as human, goat, rabbit, or sheep. The antibody may also be a chimeric antibody. The antibody may be derivatized by the attachment of one or more chemical, peptide, or polypeptide moieties known in the art. The antibody may be conjugated with a chemical moiety. The antibody may be a specific binding member.

b. Attached

“Attached” or “immobilized” as used herein to refer to a polypeptide and a solid support may mean that the binding between the polypeptide and the solid support is sufficient to be stable under conditions of binding, washing, analysis, and removal. The binding may be covalent or non-covalent. Covalent bonds may be formed directly between the polypeptide and the solid support or may be formed by a cross linker or by inclusion of a specific reactive group on either the solid support or the probe or both molecules. Non-covalent binding may be one or more of electrostatic, hydrophilic, and hydrophobic interactions. Included in non-covalent binding is the covalent attachment of a molecule, such as streptavidin, to the support and the non-covalent binding of a biotinylated polypeptide to the streptavidin. Immobilization may also involve a combination of covalent and non-covalent interactions.

c. Epitope

“Epitope” or “antigen” as used herein may mean an antigenic determinant of a polypeptide. An epitope may comprise 3 amino acids in a spatial conformation which is unique to the epitope. An epitope may comprise at least 5, 6, 7, 8, 9, or 10 amino acids. Methods of examining spatial conformation are known in the art and include, X-ray crystallography and two-dimensional nuclear magnetic resonance.

d. Fragment

“Fragment” as used herein may mean a portion of a reference peptide or polypeptide.

e. Identical

“Identical” or “identity” as used herein in the context of two or more polypeptide sequences, may mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation.

f. Indicator Reagent

“Indicator reagent” as used herein may be a composition comprising a label, which is capable of generating a measurable signal that is detectable by external means, and which may be conjugated or attached to a specific binding member for a particular polypeptide. The indicator reagent may be an antibody member of a specific binding pair for a particular polypeptide. The indicator reagent may also be a member of any specific binding pair, including hapten-anti-hapten systems such as biotin or anti-biotin, avidin, or biotin, a carbohydrate or a lectin, a complementary nucleotide sequence, an effector or a receptor molecule, an enzyme cofactor and an enzyme, or an enzyme inhibitor and an enzyme.

g. Label

“Label” or “detectable label” as used herein may mean a moiety capable of generating a signal that allows the direct or indirect quantitative or relative measurement of a molecule to which it is attached. The label may be a solid such as a microtiter plate, particle, microparticle, or microscope slide; an enzyme; an enzyme substrate; an enzyme inhibitor; coenzyme; enzyme precursor; apoenzyme; fluorescent substance; pigment; chemiluminescent compound; luminescent substance; coloring substance; magnetic substance; or a metal particle such as gold colloid; a radioactive substance such as ¹²⁵I, ¹³¹I, ³²P, ³H, ³⁵S, or ¹⁴C; a phosphorylated phenol derivative such as a nitrophenyl phosphate, luciferin derivative, or dioxetane derivative; or the like. The enzyme may be a dehydrogenase; an oxidoreductase such as a reductase or oxidase; a transferase that catalyzes the transfer of functional groups, such as an amino; carboxyl, methyl, acyl, or phosphate group; a hydrolase that may hydrolyzes a bond such as ester, glycoside, ether, or peptide bond; a lyases; an isomerase; or a ligase. The enzyme may also be conjugated to another enzyme.

The enzyme may be detected by enzymatic cycling. For example, when the detectable label is an alkaline phosphatase, a measurements may be made by observing the fluorescence or luminescence generated from a suitable substrate, such as an umbelliferone derivative. The umbelliferone derivative may comprise 4-methyl-umbellipheryl phosphate.

The fluorescent or chemiluminescent label may be a fluorescein isothiocyanate; a rhodamine derivative such as rhodamine B isothiocyanate or tetramethyl rhodamine isothiocyanate; a dancyl chloride (5-(dimethylamino)-1-naphtalenesulfonyl chloride); a dancyl fluoride; a fluorescamine (4-phenylspiro[furan-2(3H); 1ÿ-(3ÿH)-isobenzofuran]-3; 3ÿ-dione); a phycobiliprotein such as a phycocyanine or physoerythrin; an acridinium salt; a luminol compound such as lumiferin, luciferase, or aequorin; imidazoles; an oxalic acid ester; a chelate compound of rare earth elements such as europium (Eu), terbium (Tb) or samarium (Sm); or a coumarin derivative such as 7-amino-4-methylcoumarin.

The label may also be a hapten, such as adamantine, fluoroscein isothiocyanate, or carbazole. The hapten may allow the formation of an aggregate when contacted with a multi-valent antibody or (strep)avidin containing moiety. The hapten may also allow easy attachment of a molecule to which it is attached to a solid substrate.

The label may be detected by quantifying the level of a molecule attached to a detectable label, such as by use of electrodes; spectrophotometric measurement of color, light, or absorbance; or visual inspection.

h. Peptide

A “peptide” or “polypeptide” as used herein may mean a linked sequence of amino acids and may be natural, synthetic, or a modification or combination of natural and synthetic.

i. Recombinant Polypeptide

A “recombinant polypeptide” or “recombinant protein” as used herein may mean at least a polypeptide of genomic, semisynthetic or synthetic origin which by virtue of its origin or manipulation is not associated with all or a portion of the polynucleotide with which it is associated in nature or in the form of a library, or is linked to a polynucleotide other than that to which it is linked in nature. The recombinant polypeptide may not necessarily be translated from a designated nucleic acid sequence of HBV. The recombinant polypeptide may also be generated in any manner, including chemical synthesis or expression of a recombinant expression system, or isolated from HBV.

j. Solid Support

“Solid support” or “solid phase” as used herein may be the walls of wells of a reaction tray, test tubes, polystyrene beads, magnetic beads, nitrocellulose strips, membranes, microparticles such as latex particles, and others. The solid support is not critical and can be selected by one skilled in the art. Thus, latex particles, microparticles, magnetic or non-magnetic beads, membranes, plastic tubes, walls of microtiter wells, glass or silicon chips and sheep red blood cells are all suitable examples. Suitable methods for immobilizing peptides on solid supports include ionic, hydrophobic, covalent interactions and the like. The solid support may also be any material which is insoluble, or may be made insoluble by a subsequent reaction. The solid support may be chosen for its intrinsic ability to attract and immobilize the capture reagent. Alternatively, the solid support may retain an additional receptor which has the ability to attract and immobilize the capture reagent. The additional receptor may include a charged substance that is oppositely charged with respect to the capture reagent itself or to a charged substance conjugated to the capture reagent As yet another alternative, the receptor molecule may be any specific binding member which is immobilized upon (attached to) the solid support and which has the ability to immobilize the capture reagent through a specific binding reaction. The receptor molecule enables the indirect binding of the capture reagent to a solid support material before the performance of the assay or during the performance of the assay. The solid support thus may be a plastic, derivatized plastic, magnetic or non-magnetic metal, glass or silicon surface of a test tube, microtiter well, sheet, bead, microparticle, chip, and other configurations known to those of ordinary skill in the art.

It is contemplated and within the scope of the invention that the solid support also may comprise any suitable porous material with sufficient porosity to allow access by detection antibodies and a suitable surface affinity to bind antigens. Microporous structures are generally preferred, but materials with gel structure in the hydrated state may be used as well. Such useful solid supports include: natural polymeric carbohydrates and their synthetically modified, cross-linked or substituted derivatives, such as agar, agarose, cross-linked alginic acid, substituted and cross-linked guar gums, cellulose esters, especially with nitric acid and carboxylic acids, mixed cellulose esters, and cellulose ethers; natural polymers containing nitrogen, such as proteins and derivatives, including cross-linked or modified gelatins; natural hydrocarbon polymers, such as latex and rubber; synthetic polymers which may be prepared with suitably porous structures, such as vinyl polymers, including polyethylene, polypropylene, polystyrene, polyvinylchloride, polyvinylacetate and its partially hydrolyzed derivatives, polyacrylamides, polymethacrylates, copolymers and terpolymers of the above polycondensates, such as polyesters, polyamides, and other polymers, such as polyurethanes or polyepoxides; porous inorganic materials such as sulfates or carbonates of alkaline earth metals and magnesium, including barium sulfate, calcium sulfate, calcium carbonate, silicates of alkali and alkaine earth metals, aluminum and magnesium; and aluminum or silicon oxides or hydrates, such as clays, alumina, talc, kaolin, zeolite, silica gel, or glass (these materials may be used as filters with the above polymeric materials); and mixtures or copolymers of the above classes, such as graft copolymers obtained by initializing polymerization of synthetic polymers on a pre-existing natural polymer. All of these materials may be used in suitable shapes, such as films, sheets, or plates, or they may be coated onto or bonded or laminated to appropriate inert carriers, such as paper, glass, plastic films, or fabrics.

The porous structure of nitrocellulose has excellent absorption and adsorption qualities for a wide variety of reagents including monoclonal antibodies. Nylon also possesses similar characteristics and also is suitable. It is contemplated that such porous solid supports described hereinabove are preferably in the form of sheets of thickness from about 0.01 to 0.5 mm, preferably about 0.1 mm. The pore size may vary within wide limits, and is preferably from about 0.025 to 15 microns, especially from about 0.15 to 15 microns. The surfaces of such supports may be activated by chemical processes which cause covalent linkage of the antigen or antibody to the support. The irreversible binding of the antigen or antibody is obtained, however, in general, by adsorption on the porous material by poorly understood hydrophobic forces. Suitable solid supports also are described in U.S. patent application Ser. No. 227,272, which is incorporated herein by reference.

k. Specific Binding Member

“Specific binding member” as used herein may mean a member of a specific binding pair. The specific binding pair may be two different molecules where one of the molecules through chemical or physical means specifically binds to the second molecule. The specific binding member may be immunoreactive, and may be an antibody, an antigen, or an antibody/antigen complex that is capable of binding to a particular polypeptide.

l. Substantially Identical

“Substantially identical,” as used herein may mean that a first and second sequence are 50%-99% identical over a region of 8-100 or more residues.

m. Variant

“Variant” as used herein with respect to a polypeptide may mean (i) a portion of a referenced polypeptide which may be 8-100 or more amino acids; or (ii) a polypeptide that is substantially identical to a referenced polypeptide. A variant may also be a differentially processed polypeptide, such as by proteolysis, phosphorylation, or other post-translational modification.

### 2. Determining the Level of HBV Protein

Provided herein is a method for determining the level of a HBV protein in a sample.

a. Hepatitis B Virus Protein

The HBV protein may be a HBV surface antigen protein. The HBV surface antigen protein may be capable of forming part of an HBV envelope, which may expose the HBV protein on the surface of an HBV particle. The HBV surface antigen protein may comprise an epitope, which may be antigenic or a target of immune surveillance. The epitope may be a mutant epitope. The HBV surface antigen protein may also be glycosylated.

(1) Middle Hepatitis B Surface Antigen Protein (M-HBsAg)

The HBV surface antigen protein may be M-HBsAg. M-HBsAg may comprise a first portion and a second portion, and may have an overall length of about 281 amino acids. The first portion may comprise a preS2 region, and may be the first 55 amino acids of M-HBsAg. The second portion may be 226 amino acids in length and may comprise the sequence of S-HBsAg. M-HBsAg may comprise a sequence as set forth in Table 1 or a variant thereof. The first portion of M-HBsAg may also comprise an epitope. The epitope may be capable of being bound by an antibody. The antibody may be an anti-M-HBsAg-specific antibody.

TABLE 1

| SEQ ID NO | Middle HBV Surface Antigen Protein |
|---|---|
| 1 | 1 mpqnsttfhq tlqdprvral yfpaggsssg tvspaqntvs aissilsktg |
| | dpvpnmenia |
| | 61 sgllgpllvl qagffltki ltipqsldsw wtslnflggt pvclgqnsqs |
| | qisshsptcc |
| | 121 ppicpgyrwm clrrfiiflc illlclifll vlldyqgmlp vcplipgsst |
| | tstgpcktct |
| | 181 tpaqgtsmfp sccctkptdg nctcipipss wafakylwew |
| | asvrfswlsl lvpfvqwfvg |
| | 241 lsptvwlsvi wmmwywgpsl ynilspfmpl lpiffclwvy i |

(2) Small Hepatitis B Surface Antigen Protein (S-HBsAg)

The HBV surface protein may also be S-HBsAg. S-HBsAg may be about 226 amino acids in length, and may comprise a S region. The S-HBsAg may be a wild-type S-HBsAg. The S-HBsAg may comprise a sequence as set forth in Table 2, or a variant thereof. S-HBsAg may also comprise an epitope, which may be part of an “a” determinant as disclosed in U.S. Pat. No. 5,925,512 or 7,141,242, the contents of which are incorporated herein by reference. Amino acids 100-160 of S-HBsAg may also comprise an epitope.

TABLE 2

| SEQ ID NO | Small HBV Surface Antigen Protein |
|---|---|
| 2 | 1 meniasgllg pllvlqagff lltkiltipq sldswwtsln flggtpvclg |
| | qnsqsqissh |
| | 61 sptccppicp gyrwmclrrf iiflcilllc lifllvlldy |
| | qgmlpvcpli pgssttstgp |
| | 121 cktcttpaqg tsmfpsccct kptdgnctci pipsswafak |
| | ylwewasvrf swlsllvpfv |
| | 181 qwfvglsptv wlsviwmmwy wgpslynils pfmpllpiff clwvyi |

(a) Mutant S-HBsAg

S-HBsAg may also be a mutant, which may comprise a sequence that differs by at least one amino acid from a wild-type S-HBsAg. The mutation may affect the antigenicity of S-HBsAg. The antigenicity may be affected by disruption of the “a” determinant. The antigenicity of the mutant S-HBsAg may be reduced, which may allow the mutant S-HBsAg epitope to escape immune surveillance. The mutant S-HBsAg may be less detectable by an anti-HBs antibody as compared to wild-type S-HBsAg.

The mutant S-HBsAg may comprise a mutation in the “a” determinant of S-HBsAg and occur between amino acids 100-160 in the S-HBsAg sequence of any of the HBV genotypes. Key examples (but not limited to) of insertion and point mutants are described in Table 3 below. The mutant may also be a deletion. By inference, these same mutations would occur in M-HBsAg and L-HBsAg.

TABLE 3

| | | | |
|---|---|---|---|
| | | | SEQ ID NO of |
| Type of Mutation | Position | Sequence | mutant S-HBsAg |

| | | | |
|---|---|---|---|
| Insertion | After 122 | AsnThr | 4 |
| | after 122 | ArgAla | 5 |
| | After 123 | ArgGlyAla | 6 |
| | After 123 | AsnSerThrGlyProCysThrThr | 7 |
| | | (SEQ ID NO: 3) | |
| Point Mutation | 120 | Pro to Gln | 8 |
| | 123 | Thr to Ala | 9 |
| | 123 | Thr to Asn | 15 |
| | 145 | Gly to Arg | 10 |

The mutant S-HBsAg may also comprise a combination of the mutations described in Table 3, or it may comprise a mutation described in U.S. Pat. No. 5,925,512 or 7,141,242, the contents of which are incorporated herein by reference.

b. Antibody

Also provided herein is an antibody, which may be capable of binding to the HBV protein. The antibody may be bound to a label and may be attached on a solid phase. The antibody may also be a first antibody, a second antibody, or a third anti-HBV antibody. The first antibody, second antibody, and third anti-HBV antibody may all be capable of binding the HBV protein at different locations or epitopes. The first antibody, second antibody, and third anti-HBV antibody may also be bound to different labels that can be distinguished from one another. The terms “first antibody,” “second antibody,” and “third antibody” as used herein are for example purposes only. Other antibodies with the properties descried herein may also be utilized for the methods described herein.

(1) First Antibody

The first antibody may be capable of binding M-HBsAg. The first antibody may be capable of binding an epitope of M-HBsAg. The first antibody may also be capable of binding an epitope within the preS2 region of M-HBsAg. The first antibody may further be capable of distinguishing between M-HBsAg and S-HBsAg. The first antibody may not cross-react with S-HBsAg. The first antibody may also be capable of binding to M-HBsAg with higher avidity than to S-HBsAg. The first antibody may be a 50-80 or 116-34 monoclonal anti-HBV surface protein antibody or a similar antibody.

(2) Second Antibody

The second antibody may be capable of binding S-HBsAg. The second antibody may also be capable of binding the S region of M-HBsAg. The second antibody may be a H166, H57, H40, H53, or H35 monoclonal anti-HBs antibody or a similar antibody.

c. Determining the Level of HBsAg

The level of HBsAg may be determined by contacting the sample with the first antibody. The first antibody may be contacted with the sample for time and under conditions sufficient for the formation of a protein/antibody complex.

The method of determining the level of HBsAg may comprise contacting the sample with a solid support, binding the HBsAg to the solid support, and contacting HBsAg with the first antibody bound to a label. This mixture may then be incubated for a time and under conditions sufficient to form a protein/antibody complex. The level of HBsAg may be determined by detecting the measurable signal generated by the label. The level of HBsAg in the sample may be proportional to the signal generated.

The sample may be contacted with the first antibody attached to a solid support. The mixture may be incubated for a time and under conditions sufficient to form a protein/antibody complex. The mixture may then be transferred to a glass fiber matrix, which may capture the solid support. The mixture may then be contacted with an indicator reagent, which may comprise a third anti-HBV antibody bound to a label. This antibody may be monoclonal or polyclonal in nature or a mixture of either. The level of HBsAg may be determined by detecting the measurable signal generated by the label. The level of HBsAg in the sample may be proportional to the signal generated.

The sample may also be contacted with the first antibody bound to a label and attached to a solid support. The mixture may be incubated for a time and under conditions sufficient to form a protein/antibody complex. The mixture may then be contacted with an indicator reagent, which may comprise a third anti-HBs antibody bound to a label. The level of HBsAg may be determined by detecting the measurable signal generated by the label. The level of HBsAg may also be determined according to a method as described in U.S. Pat. No. 5,795,784 or 5,856,194, the contents of which are incorporated herein by reference. The level of HBsAg in the sample may be proportional to the signal generated.

The sample may further be contacted with the first antibody attached to a solid support and with an indicator reagent, which may comprise (i) a third anti-HBV antibody and (ii) biotin. The mixture may be incubated for a time and under conditions sufficient to form a protein/antibody complex. The mixture may then be transferred to a glass fiber matrix, which may capture the solid support. The mixture may then be contacted with an indicator reagent bound to a label and comprising an anti-biotin antibody or avidin. The level of HBsAg may be determined by detecting the measurable signal generated by the label. The level of HBsAg in the sample may be proportional to the signal generated.

The sample may also be contacted with the first antibody attached to a solid support and with an indicator reagent, which may comprise a third anti-HBV antibody bound to a label. The mixture may be incubated for a time and under conditions sufficient to form a protein/antibody complex. The level of HBsAg may be determined by detecting the measurable signal generated by the label. The level of HBsAg in the sample may be proportional to the signal generated.

The sample may further be contacted with the first antibody attached to a solid support. The mixture may be incubated for a time and under conditions sufficient to form a protein/antibody complex. The mixture may then be contacted with an indicator reagent, which may comprise a third anti-HBV antibody bound to a label. The mixture may be incubated for a second time and under conditions sufficient to form a protein/antibody complex. The level of HBsAg may be determined by detecting the measurable signal generated by the label. The level of HBsAg in the sample may be proportional to the signal generated.

A non-solid phase diagnostic assay may be used in the method. These assays are well-known to those of ordinary skill in the art and are considered to be within the scope of the present invention. Examples of such assays include those described in U.S. Pat. No. 5,925,512 or 7,141,242, the contents of which are incorporated herein by reference.

The label may be detected using a detection system, which may comprise a solid support. The solid support may be adapted to be used by a semi-automated or fully automated immunoanalyzer. The detection system may deliver the sample and reagents (which may comprise an antibody, a label, a buffer, or the like) to a reaction vessel, perform incubations, and optionally wash an unbound labeled polypeptide from a bound labeled polypeptide. The detection system may be automated without user intervention once the sample and reagents are inserted into the system. The automated detection system may be distinguished from a manual or less-automated system by the ability of the system to perform at least 8, 16, 64 or 128 assays in a 48-hour period without user intervention. The system may also be able to calculate the concentration or quantity of a polypeptide in the sample automatically, without the need for human calculation or input.

The detection system may also comprise a cartridge format or test strip assay. The detection system may provide unit-dose loadable assay reagents into a disposable instrument, and the unit-dose may contain all the reagents necessary to assay to detect the polypeptide. The disposable instrument may comprise a plastic housing, which may comprise a disposable membrane-like structure of nylon, nitrocellulose, or other suitable material. The sample may be preprocessed or loaded directly onto a loading zone of the disposable instrument. The sample may then optionally flow across the membrane-like structure through a plurality of zones contained on the membrane. The membrane-like structure may further comprise a detergent or lateral flow-aid. The membrane-like structure may also contain an absorbant to collect excess fluid and/or encourage lateral flow across the membrane. The detection system may comprise a multi-pack system in which each pack may comprise sufficient reagents to perform 1, 2, 4, 8, 10, or 12 assays.

The detection system may also comprise a microfluidic device designed to analyze the sample in the microliter range (e.g., less than 4 μL, 12 μL, or 50 μL). The microfluidic device may comprise a flow aids, propulsion device (which may comprise an expansion gel, wax, or gas), nanovalving, or the like to assist the transportation of the sample or assay reagents or both through the microfluidic device.

Of course, it goes without saying that any of the exemplary formats herein, and any assay or kit according to the invention can be adapted or optimized for use in automated and semi-automated systems (including those in which there is a solid phase comprising a microparticle), as described, e.g., in U.S. Pat. Nos. 5,089,424 and 5,006,309, the contents of which are incorporated herein by reference, and as, e.g., commercially marketed by Abbott Laboratories (Abbott Park, Ill.) including but not limited to Abbott's ARCHITECT®, AxSYM, IMX, PRISM, and Quantum II platforms, as well as other platforms.

Additionally, the assays and kits of the present invention optionally can be adapted or optimized for point of care assay systems, including Abbott's Point of Care (i-STAT™) electrochemical immunoassay system. Immunosensors and methods of manufacturing and operating them in single-use test devices are described, for example in U.S. Pat. No. 5,063,081 and published U.S. Patent Application Publication Nos. 20030170881, 20040018577, 20050054078, and 20060160164, the contents of which are incorporated herein by reference.

d. Determining the Level of S-HBsAg

The method may also comprise determining the level of S-HBsAg in the sample, which may be by contacting the sample with a monoclonal antibody. The level of S-HBsAg may be determined by detecting the measurable signal generated by the label. The amount of S-HBsAg in the sample may be proportional to the signal generated. The method may comprise steps similar to those described above for determining the level of HBsAg.

e. Determining the Level of M-HBsAg

The method may also comprise determining the level of M-HBsAg in the sample, which may be by contacting the sample with a monoclonal antibody. The level of S-HBsAg may be determined by detecting the measurable signal generated by the label. The amount of M-HBsAg in the sample may be proportional to the signal generated. The method may comprise steps similar to those described above for determining the level of HBsAg.

f. Determining the Level of L-HBsAg

The method may also comprise determining the level of L-HBsAg in the sample, which may be by contacting the sample with a monoclonal antibody. The level of L-HBsAg may be determined by detecting the measurable signal generated by the label. The amount of L-HBsAg in the sample may be proportional to the signal generated. The method may comprise steps similar to those described above for determining the level of HBsAg.

g. Sample

Provided herein is a sample, which may comprise the HBV protein. The sample may comprise HBsAg, and may also comprise S-HBsAg, M-HBsAg, or L-HBsAg. The sample may further comprise M-HBsAg and S-HBsAg in a known ratio, which may be in the range of 1000:1 and 1:1000.

The sample may be isolated from a patient. The sample may be a biological tissue or fluid isolated from an animal, such as a human. The sample may also be a section of tissue such as a biopsy or autopsy sample, a frozen section taken for histologic purposes, blood, plasma, serum, sputum, stool, tears, mucus, hair, or skin. The sample may also be an explant, or primary or transformed cell culture derived from an animal or patient tissue. The sample may be provided by removing a sample of cells from an animal, but may also be accomplished by using previously isolated cells (e.g., isolated by another person, at another time, and/or for another purpose). An archival tissue, such as that having treatment or outcome history, may also be used. The sample may also be blood, a blood fraction, effusion, ascitic fluid, saliva, cerebrospinal fluid, cervical secretion, vaginal secretion, endometrial secretion, gastrointestinal secretion, bronchial secretion, sputum, cell line, tissue sample, or secretion from the breast.

### 3. Detecting HBV

Provided herein is a method for detecting HBV, which may be by determining the level of a HBV protein, such as S-HBsAg, M-HBsAg, or L-HBsAg. The level of S-HBsAg, M-HBsAg, or L-HBsAg may be compared to a predetermined S-HBsAg, M-HBsAg, or L-HBsAg value, which may be indicative of HBV in the sample. A ratio of the level of two different HBV proteins may be calculated. A difference in the ratio compared to a predetermined ratio value may be indicative of HBV protein in the sample.

a. Predetermined M-HBsAg Value

The predetermined M-HBsAg value may be the level of M-HBsAg in a positive M-HBsAg control sample. The M-HBsAg control sample may comprise a known quantity of M-HBsAg. The M-HBsAg control sample may also comprise HBV. The predetermined M-HBsAg value may also be the level of M-HBsAg in a negative M-HBsAg control sample.

The predetermined M-HBsAg value may be a value at or above which the level of M-HBsAg in the sample may be indicative of the presence of HBV. The predetermined M-HBsAg value may also be a value below which the level of M-HBsAg may be indicative of the absence of HBV. The predetermined M-HBsAg value may be in the range of 1×10⁻¹² to 1×10⁻² gms.

b. Method for Detecting a Mutant S-HBsAg

Also provided herein is a method for detecting a mutant S-HBsAg, which may be by detecting HBV. A level of M-HBsAg at or above the predetermined value, and a level of S-HBsAg below a predetermined S-HBsAg value may be indicative of the presence of a mutant S-HBsAg

(1) Predetermined S-HBsAg Value

The predetermined S-HBsAg value may the level of S-HBsAg in a positive S-HbsAg control sample, which comprises a known quantity of S-HBsAg or which is known to comprise S-HBsAg. The predetermined S-HBsAg value may also be a value below which the level of S-HBsAg indicates the presence of a mutant S-HBsAg. The predetermined S-HBsAg value may be in the range of 1×10⁻¹² to 1×10⁻² gms.

c. Predetermined L-HBsAg Value

The predetermined L-HBsAg value may the level of L-HBsAg in a positive L-HBsAg control sample, which comprises a known quantity of L-HBsAg or which is known to comprise L-HBsAg. The predetermined L-HBsAg value may be in the range of 1×10⁻¹² to 1×10⁻² gms.

### 4. Kit

Provided herein is a kit, which may be used for detecting HBV. The kit may comprise the first antibody, and may also comprise the second antibody. The kit may also comprise a solid support suitable for binding proteins from a sample. The kit may further comprise a HBV composition comprising a HBV protein at a known concentration for use as a positive control. The positive control may be used to measure a predetermined value.

The kit may also comprise an additional reagent such as a buffer or salt, which may be required for promoting or preventing protein-protein interactions, or removing unbound proteins from a solid support. The kit may further comprise an agent capable of inducing a label on an antibody to generate a detectable signal. The kit may also comprise an agent capable of stopping a label from generating a signal.

The kit may also comprise one or more containers, such as vials or bottles, with each container containing a separate reagent. The kit may further comprise written instructions, which may describe how to perform or interpret an assay described herein.

The present invention has multiple aspects, illustrated by the following non-limiting examples.

### Example 1

### Detecting Hepatitis B

Materials and Methods:

Monoclonal anti-HBs antibody (H53 or 116-34) was coupled to polystyrene microparticles using the following procedure. Antibody was diluted to approximately 400 ug/ml in 15 mM MES buffer pH 4.7. Polystyrene microparticles were suspended and washed in 15 mM MES buffer pH 4.7 containing 0.01% Tween 20. Microparticles at 1% solids were activated with 0.04 mg/ml EDAC in 15 mM MES buffer pH 4.7 and anti-HBs antibody was added at a final concentration of 200 ug/ml. The mixture was incubated for 30 minutes and then washed with phosphate buffered saline pH 7.4. The particles were adjusted to a final concentration of 0.1% solids for immunoassay.

HBsAg testing was performed using different assay reagent configurations. The Abbott IMx HBsAg assay was used per the manufacturer's instructions. The reference IMx HBsAg assay (which utilizes anti-HBs H166 as a capture reagent) was compare to two test assay configurations containing either preS2 anti-HBs monoclonal 116-34 or the S-HbsAg monoclonal H53 as capture reagents. The biotin:anti-biotin conjugate detection system of IMx HBsAg was used to measure both test assay reactivity. The signal to cut-off (two time the negative control value) ratio or s/co was calculated for reference and the test assays. Samples were tested on the same day on the same IMx instrument to minimize test variation.

Results:

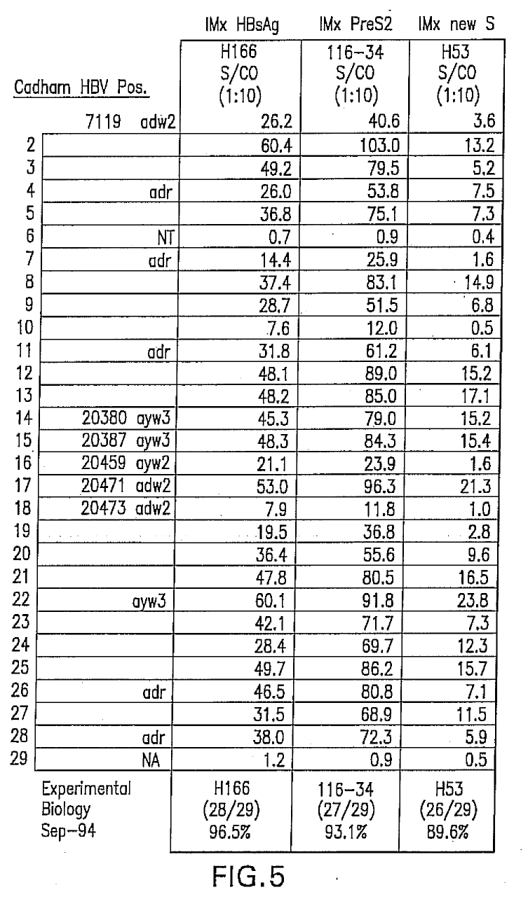

Clinical samples were tested with the reference IMx HBsAg assay and the two test assay conditions containing 116-34 or H53 on the microparticle capture phase. The clinical samples were diluted 1:10 in normal human serum to lower antigen concentration. The assay results are shown in FIG. 5. The s/co values for the 116-34 test configuration were significantly higher that the values for the reference assay using H166 for immunocapture of HBsAg or the test assay using H53 for immunocapture of HBsAg. The conclusion from this data is that a preS2 monoclonal capture format can increase microparticle assay sensitivity to wildtype forms of HBsAg.

### Example 2

### Detecting Mutant Hepatitis B

Materials and Methods:

Monoclonal anti-HBs antibody (H166, H57, H35, H53, and 116-34) was coupled to polystyrene microparticles using the following procedure. Antibody was diluted to approximately 400 ug/ml in 15 mM MES buffer pH 4.7. Polystyrene microparticles were suspended and washed in 15 mM MES buffer pH 4.7 containing 0.01% Tween 20. Microparticles at 1% solids were activated with 0.04 mg/ml EDAC in 15 mM MES buffer pH 4.7 and anti-HBs antibody was added at a final concentration of 200 ug/ml. The mixture was incubated for 30 minutes and then washed with phosphate buffered saline pH 7.4. The particles were adjusted to a final concentration of 0.1% solids for immunoassay.

HBsAg testing was performed using different assay reagent configurations. The assay configurations used either preS2 anti-HBs monoclonal 116-34 or S-HBsAg monoclonals (H166, H57, H35, H53) as capture reagents. The biotin:anti-biotin conjugate detection system of Imx HBsAg was used to measure assay reactivity. The signal to cut-off (two time the negative control value) ratio or s/co was calculated for reference and the test assays.

An HBV panel consisting of HBsAg standards at a known ng/ml concentration were tested with the different anti-HBs monoclonal coupled microparticles. In addition, clinical samples containing known HBsAg mutants were also tested. All samples were tested on the same day on the same IMx instrument to minimize test variation.

Results:

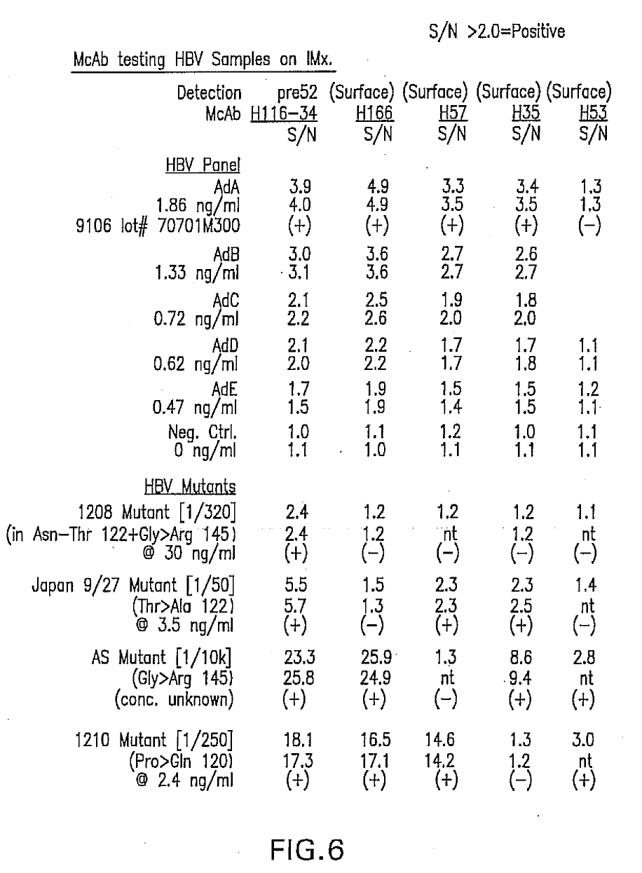

Samples were tested in the IMx HBsAg assay configuration using preS2 anti-HBs monoclonal 116-34 or S-HBsAg monoclonals (H166, H57, H35, H53) as capture reagents. The assay results are shown in FIG. 6. Overall the s/co values for the 116-34 test configuration were higher that the values for S-HBsAg monoclonals for the immunocapture of mutant forms of HBsAg. The conclusion from this data is that a preS2 monoclonal capture format can increase microparticle assay sensitivity to mutant forms of HBsAg.

### Example 3

### Detecting Mutant Hepatitis B

Materials and Methods:

HBsAg mutants were produced as previously described (Coleman et al., J Med. Virol. 59:19-24 (1999)). Briefly, surface antigen gene sequences containing different, defined mutations introduced into the same starting wildtype sequence were cloned into the XhoI and HpaI restriction sites of a proprietary expression vector. Inserted genes were verified by sequencing, and the verified expression vector was used to transiently transfect mouse L cells at 85% confluency in the presence of lipofectamine. Cell culture supernatant was monitored at day three for the expression of recombinant antigen. Initial quantitation was done using the Abbott Ausria assay because the polyclonal capture and polyclonal detection reagent format of this assay was capable of detecting all mutants. Cell culture supernatant was diluted into normal human plasma and tested for the presence of HBsAg.

Monoclonal anti-HBs (H166, H40, H57, H35, H53, and 116-34) was coated on polystyrene beads using the following procedure. Quarter inch polystyrene beads were washed three times with distilled water. Anti-HBs monoclonal antibodies were diluted to 10 microgram per ml in 0.25M citrate buffer pH 7.2. The washed beads were added to the anti-HBs solution in a capped bottle and rotated for two hours and twenty minutes at 46 deg C. The anti-HBs solution was then removed from the beads. The beads were washed with 0.25M citrate buffer pH 7.2 with 0.05% Tween-20, followed by 0.25M citrate buffer pH 7.2 alone, followed by a 0.4M citrate dihydrate 1% phosphate glass 2% sucrose buffer pH 7.2 and then air-dried.

HBsAg testing was performed using different assay reagent configurations. The Abbott Auszyme Monoclonal assay was used per the manufacturer's instructions. This reference condition was compared to the test solid phase capture beads coated with monoclonal anti-HBs that recognize either preS2 (116-34) or the “a” determinant of S-HBsAg (H166, H40, H57, H35, and H53) which were incubated with a polyclonal anti-HBs:horse radish peroxidase conjugate. The optical absorption (A) at 492 nm was measured. Samples were run in triplicate and averaged. The ability of different anti-HBs monoclonal reagents to detect the recombinant HBsAg mutants was evaluated.

Results:

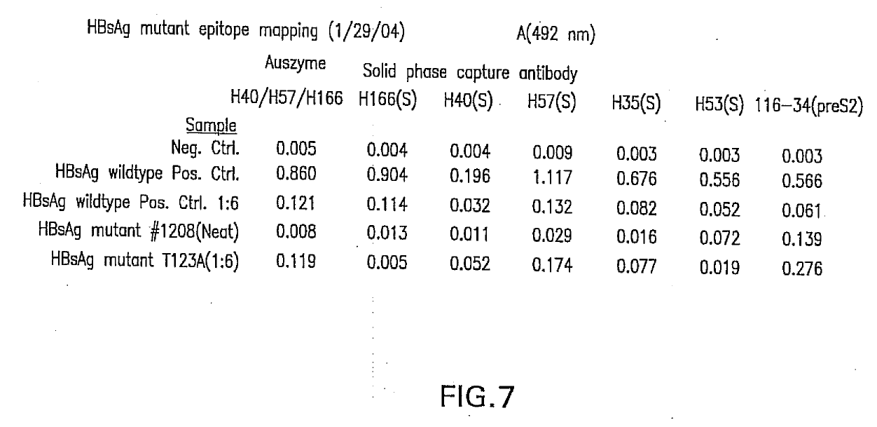

Two recombinant HBsAg mutants were evaluated in this study, HBsAg mutant 1208 which contains an insertion of Asn Thr at amino acid position 122 and HBsAg mutant T123A which contains the substitution of Thr to Ala at amino acid position 123. In addition the Auszyme positive control containing wildtype HBsAg was run at two concentrations. The Auszyme results and the anti-HBs monoclonal reactivity results are shown in FIG. 7. While all the anti-HBs coated beads recognized the wildtype positive control, they showed different reactivity for the two HBsAg mutants. The anti-HBs monoclonal with the highest A(492 nm) reading for both mutants was 116-34 which is directed against the preS2 region of M-HBsAg. The 116-34 signal was significantly more sensitive than the other anti-HBs monoclonals or the Auszyme kit signal. A monoclonal combination of H53 and 116-34 would best detect the HbsAg mutant 1208, while a monoclonal combination of H57 and 116-34 would best detect the HbsAg mutant T123A. The conclusion from this data is that a preS2 monoclonal capture format increases assay sensitivity to mutant forms of HBsAg.

1. A method for detecting hepatitis B in a test sample, comprising:
(a) providing a test sample; and
(b) contacting said test sample with a first antibody for a time and under conditions sufficient to form middle hepatitis B surface protein (M-HBsAg)/first antibody complexes, presence of said complexes indicating detection of hepatitis B present said sample.

2. The method of claim 1, wherein amino acids 1-55 of M-HBsAg comprise an epitope recognized by the first antibody.

3. The method of claim 2, wherein the first antibody does not cross-react with small hepatitis B surface protein (S-HBsAg).

4. The method of claim 1, further comprising the step of contacting said test sample with a second antibody for a time and under conditions sufficient to form small hepatitis B surface protein (S-HBsAg)/second antibody complexes, presence of said complexes indicating detection of hepatitis B present in said test sample.

5. The method of claim 4, wherein amino acids 100-160 of S-HBsAg comprise an epitope recognized by the second antibody.

6. A method for detecting a mutant S-HBsAg in a test sample comprising:
(a) providing a test sample;
(b) contacting said test sample with a first antibody for a time and under conditions sufficient to form middle hepatitis B surface protein (M-HBsAg)/first antibody complexes;
(c) contacting said test sample with a second antibody for a time and under conditions sufficient to form small hepatitis B surface protein (S-HBsAg)/second antibody complexes; and
(d) comparing level of M-HBsAg/first antibody complexes to level of S-HBsAg/second antibody complexes, wherein a reduced level of S-HBsAg/second antibody complexes to M-HBsAg/first antibody complexes indicates detection of a mutant S-HBsAg in the test sample.

7. The method of claim 6, wherein amino acids 1-55 of M-HBsAg comprise an epitope recognized by the first antibody.

8. The method of claim 6, wherein the first antibody does not cross-react with small hepatitis B surface protein (S-HBsAg).

9. The method of claim 6, wherein amino acids 100-160 of S-HBsAg comprise an epitope recognized by the second antibody.

10. A method for detecting a mutant S-HBsAg in a test sample, comprising:
(a) providing a test sample;
(b) contacting said test sample with a first antibody for a time and under conditions sufficient to form small hepatitis B surface protein (S-HBsAg)/antibody complex
wherein a reduced level of S-HBsAg/antibody complex in comparison to a control indicates detection of a mutant S-HBsAg in the test sample.

11. The method of claim 10, wherein said first antibody does not cross-react with mutant small hepatitis B surface protein (S-HBsAg).

12. The method of claim 10, further comprising the steps of
(a) contacting said test sample with a second antibody for a time and under conditions sufficient to form a small hepatitis B surface protein (S-HBs-Ag)/second antibody complex; and
(b) comparing the level of S-HBsAg/first antibody complex to level of S-HBs/second antibody complex,
wherein a difference in the ratio of the level of S-HBsAg detected by the first antibody to the level of S-HBsAg detected by the second antibody as compared to a predetermined ratio may indicate detection of mutant S-HBsAg in the test sample.

13. A kit for detecting hepatitis B comprising:
(a) at least one antibody that binds to M-HBsAg; and
(b) at least one antibody that binds to S-HBsAg.

14. The kit of claim 13, wherein amino acids 1-55 of M-HBsAg comprise an epitope recognized by the at least one antibody of (a).

15. The kit of claim 13, wherein the at least one antibody does not cross-react with S-HBsAg.

16. The kit of claim 13, wherein amino acids 100-160 of S-HBsAg comprise an epitope recognized by the at least one antibody of (b).

17. The kit of claim 13, further comprising instructions for said detection of said hepatitis B.


SEQUENCE LISTING (15 sequences)

SEQ ID NO: 1 - protein, 280 aa, Hepatitis B virus
  preS2 sequence
MPQNSTTFHQTLQDPRVRALYFPAGGSSSGTVSPAQNTVSAISSILSKTGDPVPNMENIASGLLGPLLVLQAGFFLTKILTIPQSLDSWWTSLNFLGGTPVCLGQNSQSQISSHSPTCCPPICPGYRWMCLRRFIIFLCILLLCLIFLLVLLDYQGMLPVCPLIPGSSTTSTGPCKTCTTPAQGTSMFPSCCCTKPTDGNCTCIPIPSSWAFAKYLWEWASVRFSWLSLLVPFVQWFVGLSPTVWLSVIWMMWYWGPSLYNILSPFMPLLPIFFCLWVYI

SEQ ID NO: 2 - protein, 226 aa, Hepatitis B virus
  S sequence
MENIASGLLGPLLVLQAGFFLLTKILTIPQSLDSWWTSLNFLGGTPVCLGQNSQSQISSHSPTCCPPICPGYRWMCLRRFIIFLCILLLCLIFLLVLLDYQGMLPVCPLIPGSSTTSTGPCKTCTTPAQGTSMFPSCCCTKPTDGNCTCIPIPSSWAFAKYLWEWASVRFSWLSLLVPFVQWFVGLSPTVWLSVIWMMWYWGPSLYNILSPFMPLLPIFFCLWVYI

SEQ ID NO: 3 - protein, 8 aa, Artificial Sequence
  Description of Artificial Sequence Synthetic peptide
NSTGPCTT

SEQ ID NO: 4 - protein, 228 aa, Artificial Sequence
  Description of Artificial Sequence Synthetic polypeptide
MENIASGLLGPLLVLQAGFFLLTKILTIPQSLDSWWTSLNFLGGTPVCLGQNSQSQISSHSPTCCPPICPGYRWMCLRRFIIFLCILLLCLIFLLVLLDYQGMLPVCPLIPGSSTTSTGPCKNTTCTTPAQGTSMFPSCCCTKPTDGNCTCIPIPSSWAFAKYLWEWASVRFSWLSLLVPFVQWFVGLSPTVWLSVIWMMWYWGPSLYNILSPFMPLLPIFFCLWVYI

SEQ ID NO: 5 - protein, 228 aa, Artificial Sequence
  Description of Artificial Sequence Synthetic polypeptide
MENIASGLLGPLLVLQAGFFLLTKILTIPQSLDSWWTSLNFLGGTPVCLGQNSQSQISSHSPTCCPPICPGYRWMCLRRFIIFLCILLLCLIFLLVLLDYQGMLPVCPLIPGSSTTSTGPCKRATCTTPAQGTSMFPSCCCTKPTDGNCTCIPIPSSWAFAKYLWEWASVRFSWLSLLVPFVQWFVGLSPTVWLSVIWMMWYWGPSLYNILSPFMPLLPIFFCLWVYI

SEQ ID NO: 6 - protein, 229 aa, Artificial Sequence
  Description of Artificial Sequence Synthetic polypeptide
MENIASGLLGPLLVLQAGFFLLTKILTIPQSLDSWWTSLNFLGGTPVCLGQNSQSQISSHSPTCCPPICPGYRWMCLRRFIIFLCILLLCLIFLLVLLDYQGMLPVCPLIPGSSTTSTGPCKTRGACTTPAQGTSMFPSCCCTKPTDGNCTCIPIPSSWAFAKYLWEWASVRFSWLSLLVPFVQWFVGLSPTVWLSVIWMMWYWGPSLYNILSPFMPLLPIFFCLWVYI

SEQ ID NO: 7 - protein, 234 aa, Artificial Sequence
  Description of Artificial Sequence Synthetic polypeptide
MENIASGLLGPLLVLQAGFFLLTKILTIPQSLDSWWTSLNFLGGTPVCLGQNSQSQISSHSPTCCPPICPGYRWMCLRRFIIFLCILLLCLIFLLVLLDYQGMLPVCPLIPGSSTTSTGPCKTNSTGPCTTCTTPAQGTSMFPSCCCTKPTDGNCTCIPIPSSWAFAKYLWEWASVRFSWLSLLVPFVQWFVGLSPTVWLSVIWMMWYWGPSLYNILSPFMPLLPIFFCLWVYI

SEQ ID NO: 8 - protein, 226 aa, Artificial Sequence
  Description of Artificial Sequence Synthetic polypeptide
MENIASGLLGPLLVLQAGFFLLTKILTIPQSLDSWWTSLNFLGGTPVCLGQNSQSQISSHSPTCCPPICPGYRWMCLRRFIIFLCILLLCLIFLLVLLDYQGMLPVCPLIPGSSTTSTGQCKTCTTPAQGTSMFPSCCCTKPTDGNCTCIPIPSSWAFAKYLWEWASVRFSWLSLLVPFVQWFVGLSPTVWLSVIWMMWYWGPSLYNILSPFMPLLPIFFCLWVYI

SEQ ID NO: 9 - protein, 226 aa, Artificial Sequence
  Description of Artificial Sequence Synthetic polypeptide
MENIASGLLGPLLVLQAGFFLLTKILTIPQSLDSWWTSLNFLGGTPVCLGQNSQSQISSHSPTCCPPICPGYRWMCLRRFIIFLCILLLCLIFLLVLLDYQGMLPVCPLIPGSSTTSTGPCKACTTPAQGTSMFPSCCCTKPTDGNCTCIPIPSSWAFAKYLWEWASVRFSWLSLLVPFVQWFVGLSPTVWLSVIWMMWYWGPSLYNILSPFMPLLPIFFCLWVYI

SEQ ID NO: 10 - protein, 226 aa, Artificial Sequence
  Description of Artificial Sequence Synthetic polypeptide
MENIASGLLGPLLVLQAGFFLLTKILTIPQSLDSWWTSLNFLGGTPVCLGQNSQSQISSHSPTCCPPICPGYRWMCLRRFIIFLCILLLCLIFLLVLLDYQGMLPVCPLIPGSSTTSTGPCKTCTTPAQGTSMFPSCCCTKPTDRNCTCIPIPSSWAFAKYLWEWASVRFSWLSLLVPFVQWFVGLSPTVWLSVIWMMWYWGPSLYNILSPFMPLLPIFFCLWVYI

SEQ ID NO: 11 - protein, 6 aa, Artificial Sequence
  Description of Artificial Sequence Synthetic peptide
CXNTTC

SEQ ID NO: 12 - protein, 6 aa, Artificial Sequence
  Description of Artificial Sequence Synthetic peptide
CXRATC

SEQ ID NO: 13 - protein, 7 aa, Artificial Sequence
  Description of Artificial Sequence Synthetic peptide
CXTRGAC

SEQ ID NO: 14 - protein, 12 aa, Artificial Sequence
  Description of Artificial Sequence Synthetic peptide
CXTNSTGPCTTC

SEQ ID NO: 15 - protein, 226 aa, Artificial Sequence
  Description of Artificial Sequence Synthetic polypeptide
MENIASGLLGPLLVLQAGFFLLTKILTIPQSLDSWWTSLNFLGGTPVCLGQNSQSQISSHSPTCCPPICPGYRWMCLRRFIIFLCILLLCLIFLLVLLDYQGMLPVCPLIPGSSTTSTGPCKNCTTPAQGTSMFPSCCCTKPTDGNCTCIPIPSSWAFAKYLWEWASVRFSWLSLLVPFVQWFVGLSPTVWLSVIWMMWYWGPSLYNILSPFMPLLPIFFCLWVYI